(19) United States
(12) Patent Application Publication
(10) Pub. No.: US 2026/0227357 A1
(43) Pub. Date: Aug. 6, 2026

(54) HYDROGEN DETECTION DEVICE AND METHOD FOR MANUFACTURING THE SAME

(71) Applicant: Nuvoton Technology Corporation Japan, Kyoto (JP)

(72) Inventors: Kazunari HOMMA, Kyoto (JP); Koji KATAYAMA, Kyoto (JP); Tomohiro MINAGAWA, Kyoto (JP); Ken KAWAI, Kyoto (JP); Satoru ITO, Kyoto (JP)

(21) Appl. No.: 19/576,352

(22) Filed: Mar. 24, 2026

Related U.S. Application Data
(63) Continuation of application No. PCT/JP2024/036071, filed on Oct. 9, 2024.

(30) Foreign Application Priority Data
Oct. 11, 2023 (JP) 2023-176093

(51) Int. Cl. G01N 27/12; G01N 33/00

(52) U.S. Cl. CPC G01N 27/125; G01N 27/122; G01N 33/005

(57) ABSTRACT

A hydrogen detection device includes: a bridge circuit including a first resistive element (hydrogen sensor), a second resistive element (reference element), a third resistive element (reference element), and a fourth resistive element (reference element). Each of the first to fourth resistive elements is provided on a semiconductor substrate and includes: first and second electrodes whose principal surfaces face each other; a metal oxide layer disposed in contact with the principal surfaces of the first and second electrodes; and insulating film and the like covering the second electrode and the like, and in at least the first resistive element among the first to fourth resistive elements, the insulating film and the like include an opening that is not covered by the insulating film and the like and through which an other surface of the second electrode opposite to the principal surface of the second electrode is exposed.

### CROSS REFERENCE TO RELATED APPLICATION(S)

This is a continuation application of PCT International Patent Application No. PCT/JP2024/036071 filed on Oct. 9, 2024, designating the United States of America, which is based on and claims priority of Japanese Patent Application No. 2023-176093 filed on Oct. 11, 2023. The entire disclosures of the above-identified applications, including the specifications, drawings and claims are incorporated herein by reference in their entirety.

### FIELD

The present disclosure relates to a hydrogen detection device and a method for manufacturing the same, and in particular relates to: a hydrogen detection device including a bridge circuit; and a method for manufacturing the same.

### BACKGROUND

A hydrogen detection device that includes a bridge circuit including four resistive elements has been conventionally proposed (see Patent Literature (PTL) 1, for example). It should be noted that the bridge circuit is a Wheatstone bridge circuit.

### Citation List

### Patent Literature

PTL: Japanese Unexamined Patent Application Publication No. 2019-152451

### SUMMARY

### Technical Problem

However, the hydrogen detection device disclosed in PTL 1 requires a heater and a temperature controller, and therefore needs to be improved upon.

In view of the above, the present disclosure provides: a hydrogen detection device that includes a bridge circuit, does not necessarily require a heater, and can operate stably; and a method for manufacturing the same.

### Solution to Problem

A hydrogen detection device according to an aspect of the present disclosure includes: a bridge circuit including a first resistive element, a second resistive element, a third resistive element, and a fourth resistive element, wherein one end of the first resistive element and one end of the second resistive element are connected to each other, one end of the third resistive element and one end of the fourth resistive element are connected to each other, an other end of the first resistive element and an other end of the third resistive element are connected to each other, an other end of the second resistive element and an other end of the fourth resistive element are connected to each other, each of the first resistive element, the second resistive element, the third resistive element, and the fourth resistive element is provided on a semiconductor substrate and includes: a first electrode including a principal surface and a second electrode including a principal surface, the principal surface of the first electrode and the principal surface of the second electrode facing each other; a metal oxide layer disposed in contact with the principal surface of the first electrode and the principal surface of the second electrode; and an insulating film covering the first electrode, the second electrode, and the metal oxide layer, and in at least the first resistive element among the first resistive element, the second resistive element, the third resistive element, and the fourth resistive element, the insulating film includes an opening that is not covered by the insulating film and through which an other surface of the second electrode opposite to the principal surface of the second electrode is exposed.

A manufacturing method for manufacturing a hydrogen detection device according to an aspect of the present disclosure is a manufacturing method for manufacturing a hydrogen detection device that includes a bridge circuit including a first resistive element, a second resistive element, a third resistive element, and a fourth resistive element. The manufacturing method includes: forming, on a semiconductor substrate, layered bodies for the first resistive element, the second resistive element, the third resistive element, and the fourth resistive element; and forming an opening in at least one of the layered bodies formed, wherein in the forming of layered bodies, a layered body including: a first electrode including a principal surface and a second electrode including a principal surface, the principal surface of the first electrode and the principal surface of the second electrode facing each other; a metal oxide layer disposed in contact with the principal surface of the first electrode and the principal surface of the second electrode; and an insulating film that covers the first electrode, the second electrode, and the metal oxide layer is formed as each of the layered bodies for the first resistive element, the second resistive element, the third resistive element, and the fourth resistive element, and in the forming of an opening, an opening that is not covered by the insulating film and through which an other surface of the second electrode opposite to the principal surface of the second electrode is exposed is formed in at least the insulating film of the layered body for the first resistive element.

### Advantageous Effects

The present disclosure provides: a hydrogen detection device that includes a bridge circuit, does not necessarily require a heater, and can operate stably; and a method for manufacturing the same.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is an equivalent circuit diagram of a hydrogen detection device according to Embodiment 1.

FIG. 2A is a cross-sectional view illustrating an example of a configuration of a hydrogen sensor illustrated in FIG. 1.

FIG. 2B is a top view illustrating the example of the configuration of the hydrogen sensor illustrated in FIG. 2A.

FIG. 3 is a cross-sectional view illustrating an example of a configuration of each of the reference elements illustrated in FIG. 1.

FIG. 4A is a schematic cross-sectional view illustrating an example of an overall configuration of the hydrogen detection device according to Embodiment 1.

FIG. 4B is a schematic view illustrating an example of an overall configuration of a hydrogen detection device according to a variation of Embodiment 1.

FIG. 5A is a plan view illustrating an example of the layout of wiring patterns of four resistive elements in the hydrogen detection device according to Embodiment 1 illustrated in FIG. 4A.

FIG. 5B is a plan view illustrating an example of the layout of wiring patterns of four resistive elements in the hydrogen detection device according to the variation of Embodiment 1 illustrated in FIG. 4B.

FIG. 5C is a plan view illustrating the rotational symmetry of an insulating film including an opening and the rotational symmetry of a layered structure in the hydrogen detection device according to the variation of Embodiment 1.

FIG. 6A is a flowchart illustrating a manufacturing method for manufacturing the hydrogen detection device according to Embodiment 1.

FIG. 6B is a flowchart illustrating a manufacturing method for manufacturing the hydrogen detection device according to the variation of Embodiment 1.

FIG. 7 is a schematic view illustrating a configuration of a hydrogen detection device according to a reference example.

FIG. 8A is a diagram for describing the temperature dependence of an output voltage of the hydrogen detection device according to the reference example.

FIG. 8B is a diagram for describing the temperature dependence of an output voltage of the hydrogen detection device according to Embodiment 1.

FIG. 9A is a plan view illustrating an example of the layout of the hydrogen detection device according to the reference example.

FIG. 9B is a plan view illustrating an example of the layout of the hydrogen detection device according to Embodiment 1.

FIG. 10 is an equivalent circuit diagram of a hydrogen detection device according to Embodiment 2.

FIG. 11A is a schematic cross-sectional view illustrating an example of an overall configuration of the hydrogen detection device according to Embodiment 2.

FIG. 11B is a schematic view illustrating an example of an overall configuration of a hydrogen detection device according to a variation of Embodiment 2.

FIG. 12A is a plan view illustrating an example of the layout of wiring patterns of four resistive elements in the hydrogen detection device according to Embodiment 2 illustrated in FIG. 11A.

FIG. 12B is a plan view illustrating an example of the layout of four resistive elements in the hydrogen detection device according to the variation of Embodiment 2 illustrated in FIG. 11B.

FIG. 12C is a plan view illustrating the rotational symmetry of an insulating film including an opening and the rotational symmetry of a layered structure in the hydrogen detection device according to the variation of Embodiment 2.

FIG. 13A is a flowchart illustrating a manufacturing method for manufacturing the hydrogen detection device according to Embodiment 2.

FIG. 13B is a flowchart illustrating a manufacturing method for manufacturing the hydrogen detection device according to the variation of Embodiment 2.

FIG. 14A is a diagram for describing a differential voltage change amount of the hydrogen detection device according to Embodiment 1.

FIG. 14B is a diagram for describing a differential voltage change amount of the hydrogen detection device according to Embodiment 2.

### DESCRIPTION OF EMBODIMENTS

Hereinafter, embodiments of the present disclosure will be described in detail with reference to the Drawings. It should be noted that the embodiments described below each show a specific example of the present disclosure. The numerical values, shapes, materials, constituent elements, the arrangement and connection of the constituent elements, steps, the order of the steps, etc., in the following embodiments are mere examples, and therefore do not intend to limit the present disclosure. Moreover, each drawing is not necessarily an exact depiction. In each drawing, elements that have substantially the same configuration share the same reference signs, and overlapping description thereof is omitted or simplified. Moreover, “A and B are connected to each other” means that A and B are electrically connected to each other, and includes not only a case in which A and B are directly connected to each other but also a case in which A and B are indirectly connected to each other in a state where another circuit element is interposed between A and B.

### Embodiment 1

First, a Hydrogen Detection Device According to Embodiment 1 Will be described.

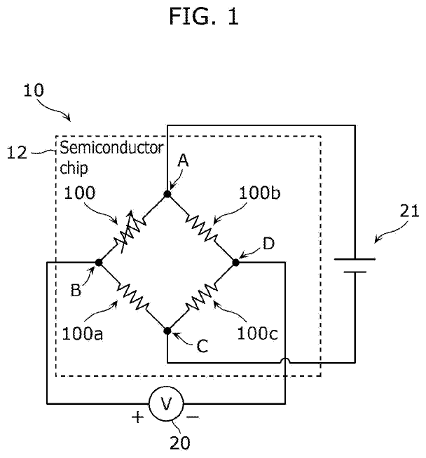

FIG. 1 is an equivalent circuit diagram of hydrogen detection device 10 according to Embodiment 1. In the present diagram, voltmeter 20 and DC voltage source 21 are also illustrated as external devices.

Hydrogen detection device 10 includes a bridge circuit including hydrogen sensor 100 that is an example of a first resistive element, reference element 100a that is an example of a second resistive element, reference element 100b that is an example of a third resistive element, and reference element 100c that is an example of a fourth resistive element. Each of one end of hydrogen sensor 100 and one end of reference element 100a is connected to terminal B, and each of one end of reference element 100b and one end of reference element 100c is connected to terminal D. Each of an other end of hydrogen sensor 100 and an other end of reference element 100b is connected to terminal A, and each of an other end of reference element 100a and an other end of reference element 100c is connected to terminal C.

Hydrogen sensor 100, reference element 100a, reference element 100b, and reference element 100c are provided on semiconductor chip 12, and have basically the same layered structure as described later. It should be noted that among the four resistive elements, only hydrogen sensor 100 has a structure in which a layered body that is sensitive to hydrogen and provided inside of hydrogen sensor 100 is exposed to the outside, and is therefore sensitive to hydrogen. Accordingly, in a hydrogen-free environment, the four resistive elements have the same resistance value. In a hydrogen-containing environment, only the resistance value of hydrogen sensor 100 decreases according to the hydrogen concentration.

A voltage of terminal B based on terminal D is measured by voltmeter 20 in a state where DC voltage from DC voltage source 21 is applied between terminal A and terminal C of hydrogen detection device 10. Since the resistance value of hydrogen sensor 100 decreases according to the hydrogen concentration, the resistance balance in the bridge circuit is disrupted, a potential difference is generated between terminal B and terminal D, and the potential difference is measured by voltmeter 20.

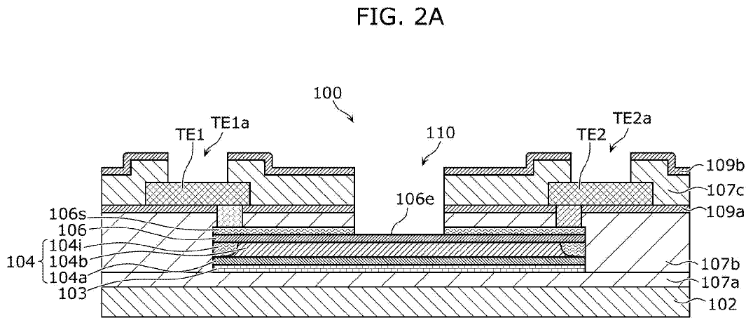

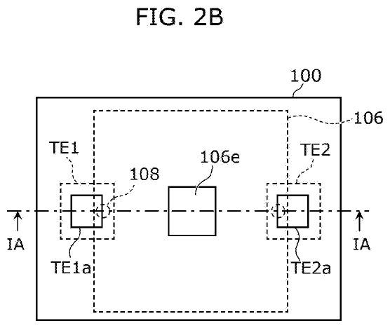

FIG. 2A is a cross-sectional view illustrating an example of a configuration of hydrogen sensor 100 illustrated in FIG. 1. FIG. 2B is a top view illustrating the example of the configuration of hydrogen sensor 100 illustrated in FIG. 2A. It should be noted that FIG. 2A schematically illustrates a cross section along line IA-IA in FIG. 2B viewed in the arrow direction.

Hydrogen sensor 100 is formed above semiconductor substrate 102 and insulating film 107a thereon, and includes, as main constituent elements: first electrode 103 including a principal surface and second electrode 106 including a principal surface, the principal surface of first electrode 103 and the principal surface of second electrode 106 facing each other; metal oxide layer 104 disposed in contact with the principal surface (i.e., upper surface) of first electrode 103 and the principal surface (i.e., lower surface) of second electrode 106; and an insulating film (insulating films 107a to 107c, 109a, and 109b) that covers first electrode 103, second electrode 106, and metal oxide layer 104. The insulating film includes opening 110 that is not covered by the insulating film and through which the other surface (i.e., upper surface) of second electrode 106 opposite to the principal surface of second electrode 106 is exposed. In the present embodiment, metal layer 106s is also removed to expose second electrode 106 through opening 110.

Hydrogen sensor 100 includes two terminals (first terminal TE1 and second terminal TE2) for connection to the outside. Each of first terminal TE1 and second terminal TE2 is connected, through via 108, to the other surface of second electrode 106. Hydrogen sensor 100 can detect hydrogen by causing current to flow in a horizontal direction in FIG. 2A, and first terminal TE1 and second terminal TE2 as one end and the other end of hydrogen sensor 100 are connected to another resistive element.

The details of each constituent element are described below.

First electrode 103 is a planar electrode and includes two surfaces. Of the two surfaces of first electrode 103, one surface (i.e., the upper surface in FIG. 2A) is in contact with metal oxide layer 104, and the other surface (i.e., the lower surface in FIG. 2A) is in contact with insulating film 107a. In FIG. 2B, first electrode 103 is in a rectangular shape of the same size as second electrode 106. First electrode 103 may be made of, for example, a material such as tungsten, nickel, tantalum, titanium, aluminum, tantalum nitride, or titanium nitride, which has a standard electrode potential lower than that of metals included in metal oxides. The higher the standard electrode potential, the less tendency towards oxidation a metal has. First electrode 103 in FIG. 2A is formed of, for example, transition metal nitride such as tantalum nitride (TaN) or titanium nitride (TiN), or a layered structure thereof.

Metal oxide layer 104 is disposed between the principal surface of first electrode 103 and the principal surface of second electrode 106 facing each other, includes a metal oxide serving as a gas-sensitive resistance film, and has a resistance value that reversibly changes according to the presence and absence of hydrogen-containing gas in gas that is in contact with second electrode 106. It is sufficient so long as metal oxide layer 104 has a property that enables its resistance to change according to hydrogen. As the base metal of metal oxide layer 104, at least one of the following may be selected: aluminum (Al) and transition metals such as tantalum (Ta), hafnium (Hf), titanium (Ti), zirconium (Zr), niobium (Nb), tungsten (W), nickel (Ni), and iron (Fe).

Metal oxide layer 104 illustrated in FIG. 2A includes: first layer 104a that is in contact with first electrode 103; second layer 104b that is in contact with first layer 104a and second electrode 106; and insulating isolation layer 104i. The degree of oxygen deficiency of second layer 104b is lower than that of first layer 104a. For example, first layer 104a is TaOₓ. Second layer 104b is Ta₂O₅ whose degree of oxygen deficiency is lower than that of first layer 104a. Moreover, metal oxide layer 104 includes insulating isolation layer 104i at the perimeter of first electrode 103 in plan view.

Here, the plan view is the plan view of semiconductor substrate 102, and means viewing hydrogen sensor 100 according to the present disclosure from a viewpoint in the layer-stacking direction in FIG. 2A; in other words, viewing from a viewpoint in the direction normal to any of the surfaces of, for example, first electrode 103 and second electrode 106 that are planar. For example, the plan view refers to viewing the top surface of hydrogen sensor 100 illustrated in FIG. 2B.

The resistance value of metal oxide layer 104 decreases according to hydrogen-containing gas that is in contact with second electrode 106 (i.e., the resistance value of metal oxide layer 104 decreases as the amount of the hydrogen-containing gas increases). Specifically, when hydrogen-containing gas is present in detection-target gas, hydrogen atoms are dissociated from the hydrogen-containing gas at second electrode 106. The hydrogen atoms dissociated enter metal oxide layer 104 and form an impurity level. Particularly, the hydrogen atoms dissociated concentrate on the vicinity of the interface with second electrode 106 and decrease the apparent thickness of second layer 104b. As a result, the resistance value of metal oxide layer 104 decreases.

Second electrode 106 is a planar electrode that is capable of dissociating hydrogen and includes two surfaces. Of the two surfaces of second electrode 106, one surface (i.e., the lower surface in FIG. 2A) is in contact with metal oxide layer 104, and the other surface (i.e., the upper surface in FIG. 2A) is in contact with metal layer 106s and the outside air. Second electrode 106 includes, in opening 110, exposed portion 106e that is exposed to the outside air. Second electrode 106 is made of a material having a function of catalyzing dissociation of hydrogen atoms from gas molecules having the hydrogen atoms, for example: noble metal such as platinum (Pt), iridium (Ir), or palladium (Pd); or nickel (Ni); or an alloy containing at least one of these. It is assumed that second electrode 106 in FIG. 2A is platinum (Pt). Two terminals, namely, first terminal TE1 and second terminal TE2 are connected to second electrode 106.

First terminal TE1 is connected to second electrode 106 through via 108. Second terminal TE2 is connected to second electrode 106 through via 108. First terminal TE1 and second terminal TE2 are connected, through openings TE1a and TE2a, to an external detection circuit (here, reference element 100a and reference element 100b) that drives hydrogen sensor 100.

As illustrated in FIG. 2B, first terminal TE1 and second terminal TE2 are arranged at positions between which exposed portion 106e is provided in the plan view of second electrode 106. Because of this arrangement, application of a predetermined voltage between first terminal TE1 and second terminal TE2 causes passage of current through exposed portion 106e of second electrode 106, that is, causes current to flow through exposed portion 106e. The passage of current through exposed portion 106e of second electrode 106 is considered to activate hydrogen dissociation by exposed portion 106e. It should be noted that the predetermined voltage may be voltages that are opposite to each other in polarity.

In hydrogen sensor 100, the resistance value between first terminal TE1 and second terminal TE2 changes when gas molecules containing hydrogen atoms come into contact with exposed portion 106e during the passage of current through exposed portion 106e. By the above-described detection circuit detecting this change in resistance value, gas molecules containing low-concentration hydrogen atoms are detected.

It should be noted that insulating films 107a to 107c, and insulating films 109a and 109b that cover main components of hydrogen sensor 100 are each formed of a silicon oxide film, a silicon nitride film, or the like.

Moreover, metal layer 106s is provided on the upper surface of second electrode 106 excluding opening 110. Metal layer 106s is made of, for example, TiAlN, and is formed as an etching stopper for forming a via, but is not essential.

It should be noted that although an example in which metal oxide layer 104 has a two-layer configuration including first layer 104a made of TaOₓ and second layer 104b made of Ta₂O₅ whose degree of oxygen deficiency is low has been illustrated in FIG. 2A, metal oxide layer 104 may have a single layer configuration including a layer made of TaOₓ or Ta₂O₅ whose degree of oxygen deficiency is low.

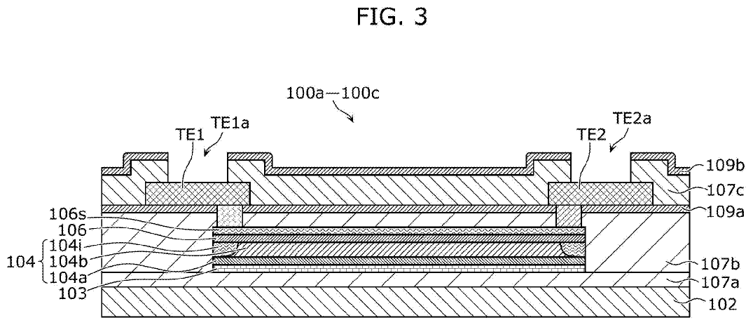

FIG. 3 is a cross-sectional view illustrating an example of a configuration of each of reference elements 100a to 100c illustrated in FIG. 1. As can be seen by comparing the present diagram and FIG. 2A, reference elements 100a to 100c are each equivalent to one in which no opening 110 is provided (i.e., opening 110 is closed) in hydrogen sensor 100 illustrated in FIG. 2A. In other words, reference elements 100a to 100c each include, as main constituent elements: first electrode 103 including a principal surface and second electrode 106 including a principal surface, the principal surface of first electrode 103 and the principal surface of second electrode 106 facing each other; metal oxide layer 104 disposed in contact with the principal surface of first electrode 103 and the principal surface of second electrode 106; and an insulating film (insulating films 107a to 107c, 109a, and 109b) that covers first electrode 103, second electrode 106, and metal oxide layer 104. The insulating film does not include an opening that is not covered by the insulating film and through which the other surface of second electrode 106 opposite to the principal surface of second electrode 106 is exposed.

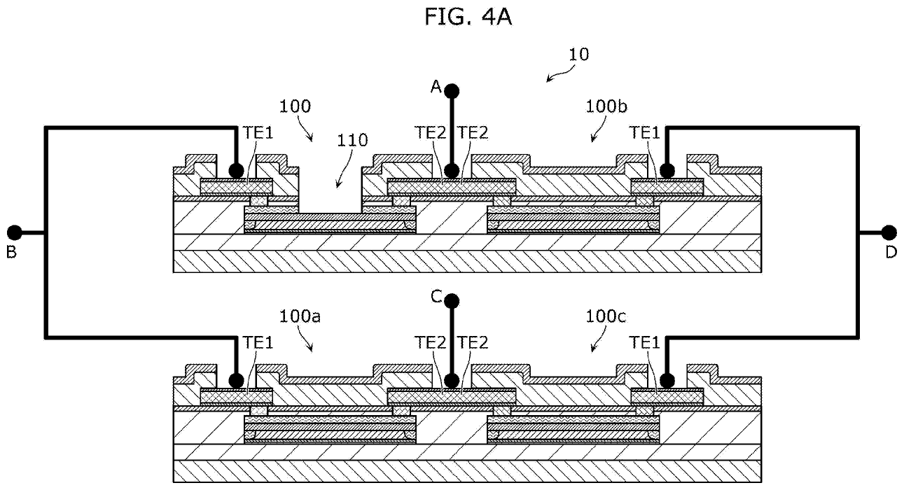

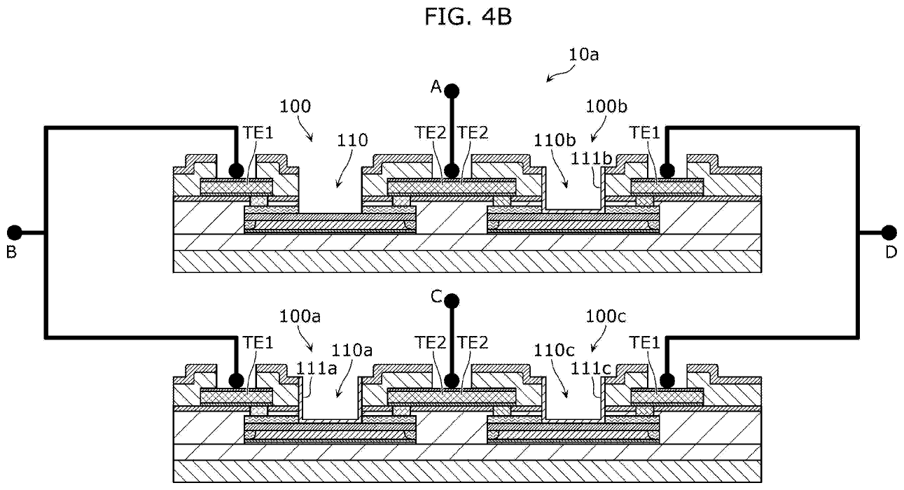

FIG. 4A is a schematic cross-sectional view illustrating an example of an overall configuration of hydrogen detection device 10 according to Embodiment 1. As illustrated in the present diagram, the feature of hydrogen detection device 10 is that four resistive elements (hydrogen sensor 100 and reference elements 100a to 100c) included in a bridge circuit are provided on semiconductor chip 12, and in particular, that the spacing between the four resistive elements that have basically the same configuration except for opening 110 of hydrogen sensor 100 is small in plan view, for example, less than or equal to 2000 μm.

First terminal TE1 (i.e., one end) of hydrogen sensor 100 and first terminal TE1 (i.e., one end) of reference element 100a are connected as terminal B, first terminal TE1 (i.e., one end) of reference element 100b and first terminal TE1 (i.e., one end) of reference element 100c are connected as terminal D, second terminal TE2 (i.e., the other end) of hydrogen sensor 100 and second terminal TE2 (i.e., the other end) of reference element 100b are connected as terminal A, and second terminal TE2 (i.e., the other end) of reference element 100a and second terminal TE2 (i.e., the other end) of reference element 100c are connected as terminal C.

FIG. 4B is a schematic view illustrating an example of an overall configuration of hydrogen detection device 10a according to a variation of Embodiment 1. The difference from hydrogen detection device 10 according to Embodiment 1 illustrated in FIG. 4A is that, in hydrogen detection device 10a according to the present variation, openings 110a to 110c similar to opening 110 of hydrogen sensor 100 are formed in reference elements 100a to 100c in the same manufacturing process and then the inner side surfaces and the bottom surfaces of openings 110a to 110c are covered by hydrogen impermeable films 111a to 111c. It should be noted that each of hydrogen impermeable films 111a to 111c is a film that does not allow hydrogen to pass through, and is, for example, a film made of a silicon oxide film, a silicon nitride film, a silicon oxynitride film, or the like.

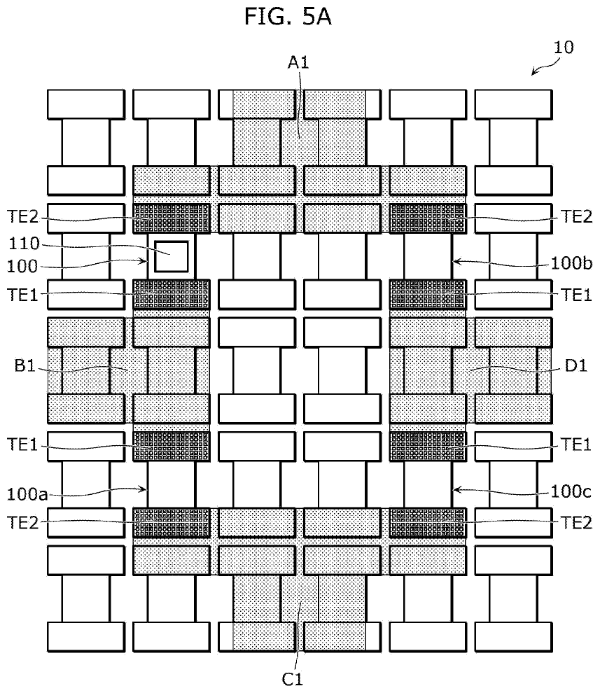

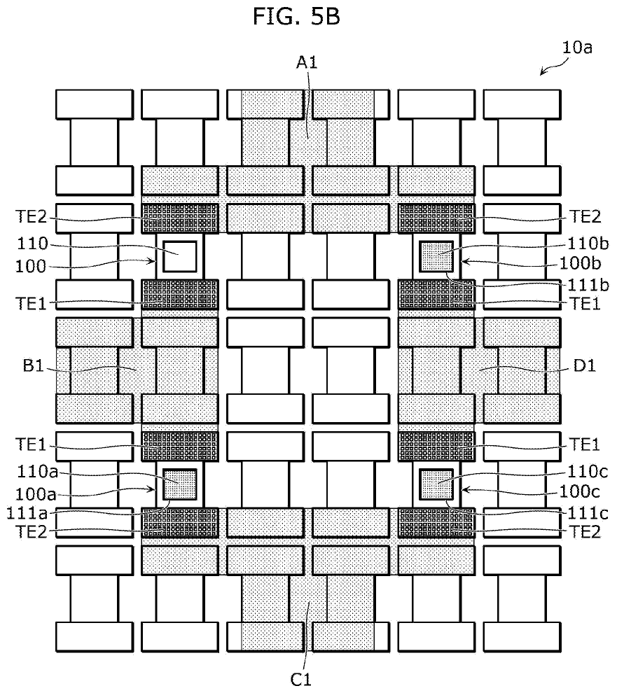

FIG. 5A is a plan view illustrating an example of the layout of wiring patterns of four resistive elements in hydrogen detection device 10 according to Embodiment 1 illustrated in FIG. 4A. The present plan view illustrates: wiring pattern A1 that connects second terminal TE2 of hydrogen sensor 100 including opening 110 and second terminal TE2 of reference element 100b without an opening, and connects to terminal A (not illustrated) of the bridge circuit; wiring pattern B1 that connects first terminal TE1 of hydrogen sensor 100 including opening 110 and first terminal TE1 of reference element 100a without an opening, and connects to terminal B (not illustrated) of the bridge circuit; wiring pattern C1 that connects second terminal TE2 of reference element 100a without an opening and second terminal TE2 of reference element 100c without an opening, and connects to terminal C (not illustrated) of the bridge circuit; and wiring pattern D1 that connects first terminal TE1 of reference element 100c without an opening and first terminal TE1 of reference element 100b without an opening, and connects to terminal D (not illustrated) of the bridge circuit.

FIG. 5B is a plan view illustrating an example of the layout of wiring patterns of four resistive elements in hydrogen detection device 10a according to the variation of Embodiment 1 illustrated in FIG. 4B. The present plan view illustrates: wiring pattern A1 that connects second terminal TE2 of hydrogen sensor 100 including opening 110 and second terminal TE2 of reference element 100b including opening 110b covered by hydrogen impermeable film 111b, and connects to terminal A (not illustrated) of a bridge circuit; wiring pattern B1 that connects first terminal TE1 of hydrogen sensor 100 including opening 110 and first terminal TE1 of reference element 100a including opening 110a covered by hydrogen impermeable film 111a, and connects to terminal B (not illustrated) of the bridge circuit; wiring pattern C1 that connects second terminal TE2 of reference element 100a including opening 110a covered by hydrogen impermeable film 111a and second terminal TE2 of reference element 100c including opening 110c covered by hydrogen impermeable film 111c, and connects to terminal C (not illustrated) of the bridge circuit; and wiring pattern D1 that connects first terminal TE1 of reference element 100c including opening 110c covered by hydrogen impermeable film 111c and first terminal TE1 of reference element 100b including opening 110b covered by hydrogen impermeable film 111b, and connects to terminal D (not illustrated) of the bridge circuit.

In any of the layout examples of FIG. 5A and FIG. 5B, the four resistive elements form a rectangular shape in the plan view of semiconductor substrate 102 and are arranged at positions corresponding to the four sides of the rectangular shape, and by the high-density integration, it is possible to reduce the size of the hydrogen detection device and to enhance the performance of the bridge circuit by unifying the temperature environment.

FIG. 5C is a plan view illustrating the rotational symmetry of opening 110 of insulating film 107b and the like and the rotational symmetry of layered structure 106a and the like in each of hydrogen detection devices 10d and 10e according to the variation of Embodiment 1. More specifically, (a) of FIG. 5C illustrates the rotational symmetry of opening 110 of insulating film 107b and the like and the rotational symmetry of layered structure 106a and the like when layered structure 106a and the like do not include an opening, and in contrast, (b) of FIG. 5C illustrates the rotational symmetry of opening 110 of insulating film 107b and the like and the rotational symmetry of layered structure 106a and the like when layered structure 106a and the like are films that include, at their center, opening 106a1 that is an electrically insulated area. It should be noted that FIG. 5C corresponds to a variation of the layout examples illustrated in FIG. 5A and FIG. 5B. Moreover, layered structure 106a and the like are layered structure films (103, 104, and 106) included in a sensor element, and insulating film 107b and the like are insulating films (insulating films 107a to 107c, 109a, and 109b) that cover hydrogen sensor 100.

In any of (a) and (b) of FIG. 5C, layered structure 106a and opening 110 of insulating film 107b and the like are arranged in a four-fold rotational symmetry pattern (i.e., a rotationally symmetric pattern with 4-fold symmetry) in the plan view of semiconductor substrate 102. In contrast, opening 110 of hydrogen sensor 100 is positioned to result in rotational symmetry in the plan view of semiconductor substrate 102. Thus, by arranging layered structure 106a and opening 110 of insulating film 107b and the like in a four-fold rotational symmetry pattern, the size of each of hydrogen detection devices 10 and 10a can be made small.

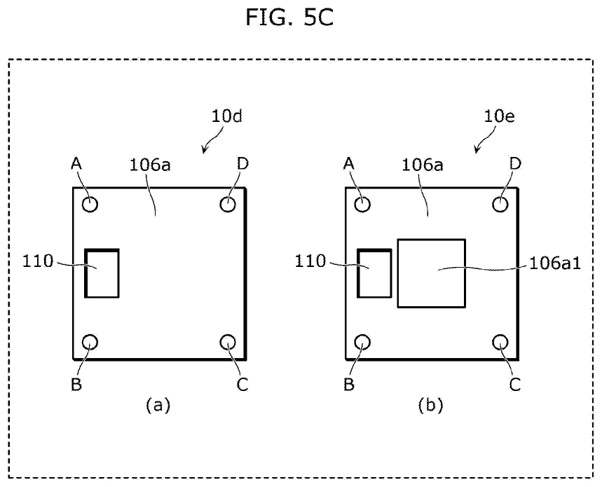

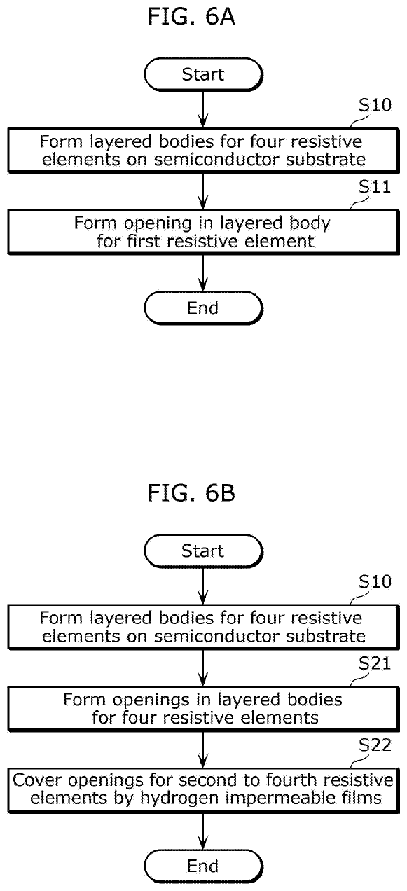

FIG. 6A is a flowchart illustrating a manufacturing method for manufacturing hydrogen detection device 10 according to Embodiment 1. First, film formation and photolithography (pattern transferring and etching) for forming layered bodies for four resistive elements (hydrogen sensor 100 and reference elements 100a to 100c) are repeatedly performed on semiconductor substrate 102 to form layered bodies each including, from the bottom: semiconductor substrate 102; insulating film 107a as an inter-layer insulating film containing, for example, plasma tetra ethoxy silane (P-TEOS); first electrode 103 containing, for example, TaN or TiN; metal oxide layer 104 including, for example, a layered body of Ta₂O₅ and TaO_{1.5}; second electrode 106 containing, for example, Pt; metal layer 106s containing, for example, TiAlN; insulating film 107b as an inter-layer insulating film containing, for example, P-TEOS; insulating film 109a as a protective film containing, for example, plasma silicon oxynitride film (P-SiON); first terminal TE1 and second terminal TE2 as electrodes each containing, for example, Au; insulating film 107c as an inter-layer insulating film containing, for example, high density plasma nitrogen doped glass (HDP-NSG); and insulating film 109b as a protective film containing, for example, P-SiON (layered body forming step S10). By this step, finished products of reference elements 100a to 100c and an intermediate product of hydrogen sensor 100 in which opening 110 has not yet been formed are manufactured.

Next, photolithography (pattern transferring and etching) is performed on the layered body for the first resistive element (i.e., the intermediate product of hydrogen sensor 100) to remove, in a rectangular shape, part of metal layer 106s, insulating film 107b, insulating film 109a, insulating film 107c, and insulating film 109b so that at least part of the upper surface of second electrode 106 is exposed, and thus opening 110 of hydrogen sensor 100 is formed (opening forming step S11). By this step, hydrogen sensor 100 is manufactured and the manufacture of hydrogen detection device 10 is completed.

FIG. 6B is a flowchart illustrating a manufacturing method for manufacturing hydrogen detection device 10a according to the variation of Embodiment 1. First, film formation and photolithography (pattern transferring and etching) for forming layered bodies for four resistive elements (hydrogen sensor 100 and reference elements 100a to 100c) are repeatedly performed on semiconductor substrate 102 to form layered bodies for hydrogen sensor 100 and reference elements 100a to 100c (layered body forming step S10). This step is the same as layered body forming step S10 shown in FIG. 6A. In the present variation, by this step, intermediate products of hydrogen sensor 100 and reference elements 100a to 100c in which openings 110 and 110a to 110c and hydrogen impermeable films 111a to 111c have not yet been formed are formed.

Next, photolithography (pattern transferring and etching) is performed on each of the layered bodies manufactured for the four resistive elements (i.e., the four intermediate products manufactured) to remove, in a rectangular shape, part of metal layer 106s, insulating film 107b, insulating film 109a, insulating film 107c, and insulating film 109b so that at least part of the upper surface of second electrode 106 is exposed, and thus openings 110 and 110a to 110c of hydrogen sensor 100 and reference elements 100a to 100c are formed (opening forming step S21). By this step, the manufacture of hydrogen sensor 100 is completed.

Finally, the inner side surfaces and the bottom surfaces of the openings for the second to fourth resistive elements (i.e., openings 110a to 110c formed for reference elements 100a to 100c) are covered by hydrogen impermeable films 111a to 111c each containing, for example, P-SiON (hydrogen impermeable film forming step S22). By this step, the manufacture of reference elements 100a to 100c provided with openings 110a to 110c including the inner side surfaces and the bottom surfaces covered by hydrogen impermeable films 111a to 111c is completed. It should be noted that the formation of hydrogen impermeable films 111a to 111c may be performed in the same process as the formation of insulating film 109b (i.e., film formation using the same material as that of insulating film 109b), or in an independent process. By this step, the manufacture of hydrogen detection device 10a according to the variation of Embodiment 1 is completed.

Next, the characteristics of hydrogen detection device 10 according to Embodiment 1 configured as described above will be described.

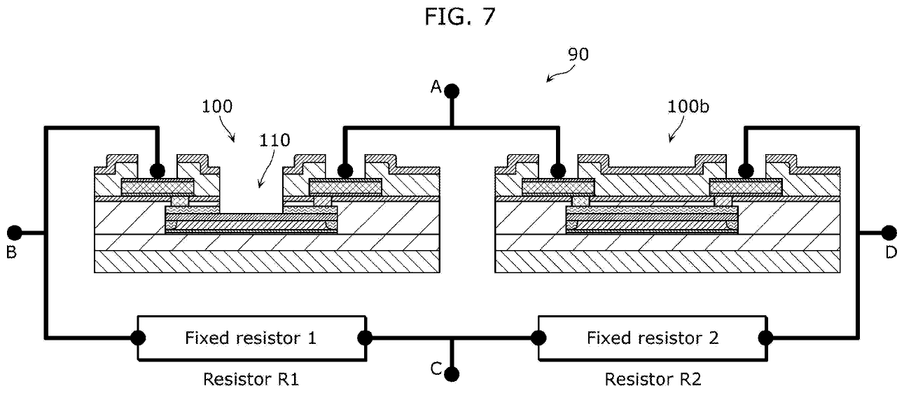

It should be noted that for convenience of explanation, hydrogen detection device 90 according to a reference example, which serves as a basis for comparison, will first be described. FIG. 7 is a schematic view illustrating a configuration of hydrogen detection device 90 according to the reference example. Hydrogen detection device 90 is a hydrogen detection device disclosed in the international application (Application Number: PCT/JP 2023/024224, Filing Date: June 29, 2023) by the inventors of the present application, and has a configuration in which reference elements 100a and 100c in hydrogen detection device 10 disclosed in FIG. 4A of the present application are replaced with resistors R1 and R2, such as polysilicon resistors, having fixed resistance values, respectively.

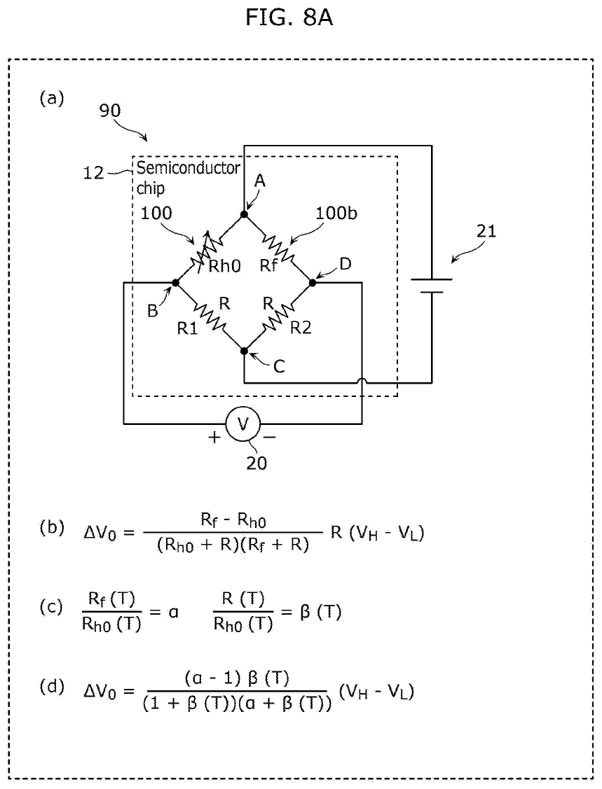

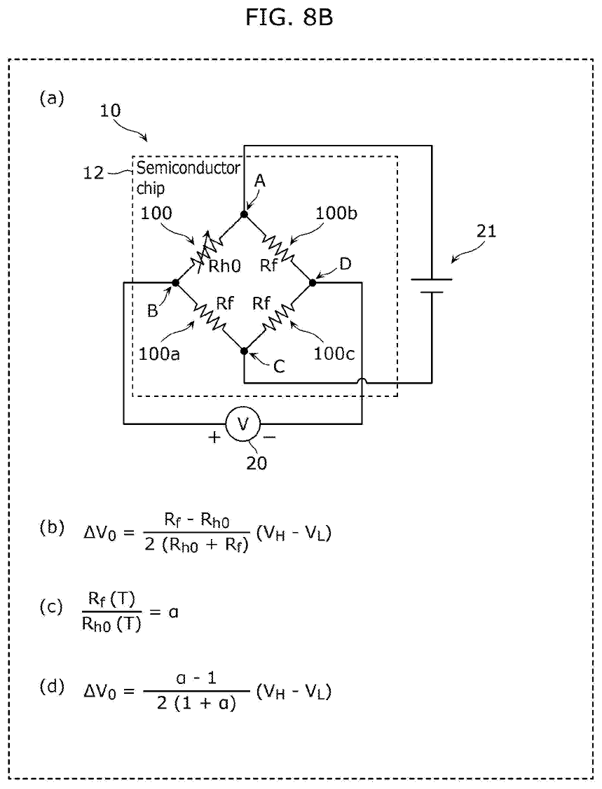

FIG. 8A is a diagram for describing the temperature dependence of an output voltage of hydrogen detection device 90 according to the reference example. (a) of FIG. 8A illustrates a bridge circuit configuration of hydrogen detection device 90 according to the reference example, and (b) to (d) of FIG. 8A show equations for explaining output voltage ΔVo (i.e., potential difference between terminal B and terminal D). In FIG. 8A, Rh0 denotes a resistance value of hydrogen sensor 100, Rf denotes a resistance value of reference element 100b, R denotes a resistance value of each of resistors R1 and R2, VH denotes a positive potential of DC voltage source 21, VL denotes a negative potential of DC voltage source 21, T denotes an environmental temperature, α denotes the ratio of the resistance value of reference element 100b to the resistance value of hydrogen sensor 100 at temperature T, and β(T) denotes the ratio of the resistance value of resistor R1 or resistor R2 to the resistance value of hydrogen sensor 100 at temperature T.

As illustrated in (a) of FIG. 8A, hydrogen detection device 90 according to the reference example outputs, as an output voltage, potential difference ΔVo between the potential at terminal B, which is a connection point between hydrogen sensor 100 having resistance value Rh0 and resistor R1 having resistance value R, and the potential at terminal D, which is a connection point between reference element 100b having resistance value Rf and resistor R2 having resistance value R. Here, since hydrogen sensor 100 and reference element 100b have the same layered structure, resistance values Rh0 and Rf also have the same temperature dependence; however, since resistors R1 and R2 each have a structure different from that of hydrogen sensor 100 and reference element 100b, resistance value R of each of resistors R1 and R2 has temperature dependence different from that of resistance values Rh0 and Rf of hydrogen sensor 100 and reference element 100b.

In hydrogen detection device 90 according to the reference example, output voltage ΔVo is represented by the equation shown in (b) of FIG. 8A. Here, as shown in (c) of FIG. 8A, when α (which is a constant for the reason described above) denotes the ratio of resistance value Rf(T) of reference element 100b to resistance value Rh0(T) of hydrogen sensor 100 at temperature T and β(T) (which has temperature dependence for the reason described above) denotes the ratio of resistance value R(T) of resistor R1 or R2 to resistance value Rh0(T) of hydrogen sensor 100 at temperature T, by substituting these into the equation shown in (b) of FIG. 8A, the equation for output voltage ΔVo of hydrogen detection device 90 according to the reference example is derived as shown in (d) of FIG. 8A. As can be seen from the fact that the equation shown in (d) of FIG. 8A includes β(T), which has temperature dependence, output voltage ΔVo of hydrogen detection device 90 according to the reference example has temperature dependence.

FIG. 8B is a diagram for describing the temperature dependence of an output voltage of hydrogen detection device 10 according to Embodiment 1. It should be noted that the content shown in the present diagram also applies to hydrogen detection device 10a according to the variation of Embodiment 1.

(a) of FIG. 8B illustrates a bridge circuit configuration of hydrogen detection device 10 according to Embodiment 1, and (b) to (d) of FIG. 8B show equations for explaining output voltage ΔVo (i.e., potential difference between terminal B and terminal D). In FIG. 8B, Rh0 denotes a resistance value of hydrogen sensor 100, Rf denotes a resistance value of each of reference elements 100a to 100c, VH denotes a positive potential of DC voltage source 21, VL denotes a negative potential of DC voltage source 21, T denotes an environmental temperature, and α denotes the ratio of the resistance value of any one of reference elements 100a to 100c to the resistance value of hydrogen sensor 100 at temperature T.

As illustrated in (a) of FIG. 8B, hydrogen detection device 10 according to Embodiment 1 outputs, as an output voltage, potential difference ΔVo between the potential at terminal B, which is a connection point between hydrogen sensor 100 having resistance value Rh0 and reference element 100a having resistance value Rf, and the potential at terminal D, which is a connection point between reference element 100b having resistance value Rf and reference element 100c having resistance value Rf. Here, since hydrogen sensor 100 and reference elements 100a to 100c have the same layered structure, their resistance values Rh0 and Rf also have the same temperature dependence.

In hydrogen detection device 10 according to Embodiment 1, output voltage ΔVo is represented by the equation shown in (b) of FIG. 8B. Here, as illustrated in (c) of FIG. 8B, when α (which is a constant for the reason described above) denotes the ratio of resistance value Rf(T) of reference element 100b to resistance value Rh0(T) of hydrogen sensor 100 at temperature T, by substituting it into the equation shown in (b) of FIG. 8B, the equation for output voltage ΔVo of hydrogen detection device 10 according to Embodiment 1 is derived as shown in (d) of FIG. 8B. As can be seen from the fact that the equation shown in (d) of FIG. 8B does not include β(T), which has temperature dependence, unlike the equation of hydrogen detection device 90 according to the reference example shown in (d) of FIG. 8A, output voltage ΔVo of hydrogen detection device 10 according to Embodiment 1 does not have temperature dependence, unlike hydrogen detection device 90 according to the reference example. In other words, in hydrogen detection device 10 according to Embodiment 1, output voltage ΔVo resulting from cancellation of the temperature dependence of the four resistive elements can be obtained.

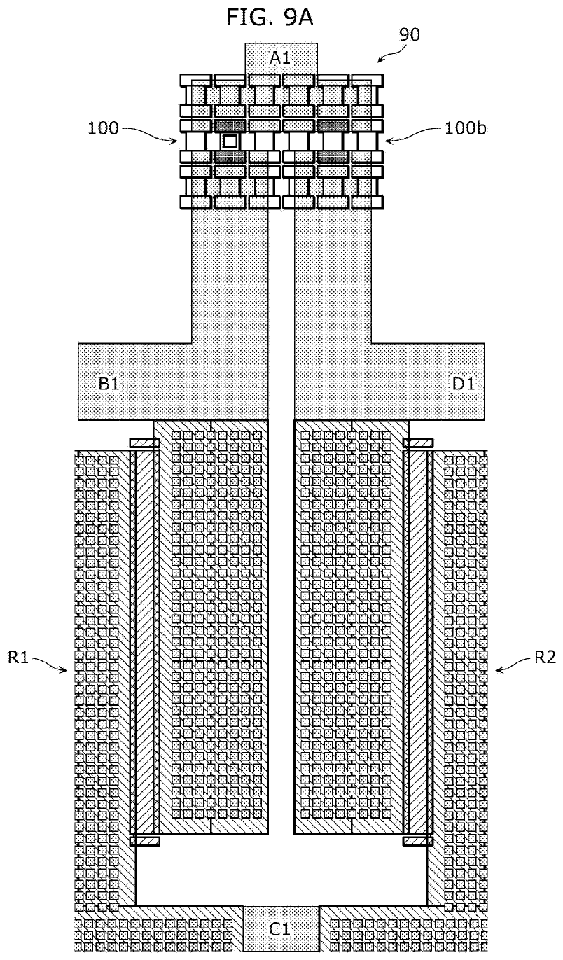

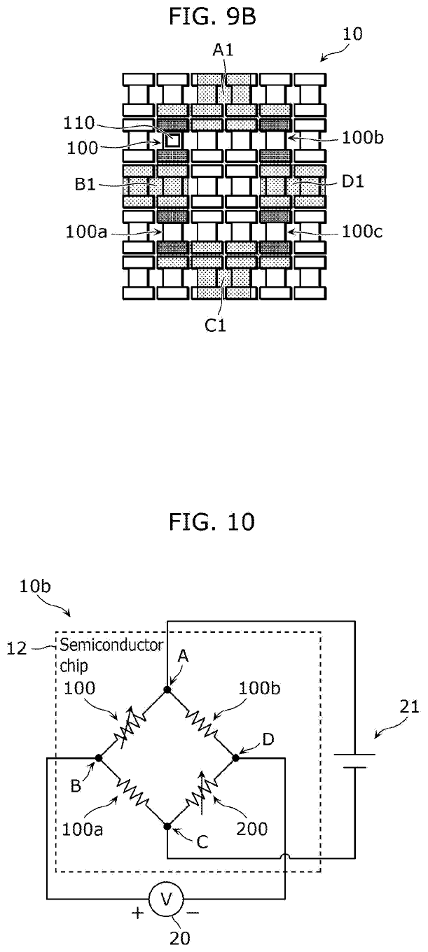

FIG. 9A is a plan view illustrating an example of the layout of hydrogen detection device 90 according to the reference example. As illustrated in the present diagram, because resistors R1 and R2 included in hydrogen detection device 90 according to the reference example are fixed resistors such as polysilicon resistors, resistors R1 and R2 need an area several times or more larger compared to hydrogen sensor 100 and reference element 100b each including metal oxide layer 104 of which resistance is variable.

FIG. 9B is a plan view illustrating an example of the layout of hydrogen detection device 10 according to Embodiment 1. As illustrated in the present diagram, the four resistive elements (hydrogen sensor 100 and reference elements 100a to 100c) included in hydrogen detection device 10 according to Embodiment 1 are layered bodies that can be finely formed. It should be noted that the content shown in the present diagram also applies to hydrogen detection device 10a according to the variation of Embodiment 1.

As can be seen by comparing FIG. 9B and FIG. 9A, the layout area of hydrogen detection device 10 according to Embodiment 1 is one fifth or less of the layout area of hydrogen detection device 90 according to the reference example. In other words, it can be seen that hydrogen detection device 10 according to Embodiment 1 has a structure suitable for miniaturization.

As described above, each of hydrogen detection devices 10 and 10a according to Embodiment 1 and a variation thereof includes: a bridge circuit including a first resistive element (hydrogen sensor 100), a second resistive element (reference element 100a), a third resistive element (reference element 100b), and a fourth resistive element (reference element 100c), wherein one end of the first resistive element and one end of the second resistive element are connected to each other, one end of the third resistive element and one end of the fourth resistive element are connected to each other, an other end of the first resistive element and an other end of the third resistive element are connected to each other, an other end of the second resistive element and an other end of the fourth resistive element are connected to each other, each of the first resistive element, the second resistive element, the third resistive element, and the fourth resistive element is provided on semiconductor substrate 102 and includes: first electrode 103 including a principal surface and second electrode 106 including a principal surface, the principal surface of first electrode 103 and the principal surface of second electrode 106 facing each other; metal oxide layer 104 disposed in contact with the principal surface of first electrode 103 and the principal surface of second electrode 106; and insulating film 107b and the like covering first electrode 103, second electrode 106, and metal oxide layer 104, and in at least the first resistive element among the first resistive element, the second resistive element, the third resistive element, and the fourth resistive element, insulating film 107b and the like include opening 110 that is not covered by insulating film 107b and the like and through which an other surface of second electrode 106 opposite to the principal surface of second electrode 106 is exposed.

Accordingly, the four resistive elements included in the bridge circuit are resistive elements that have basically the same layered structure, and are provided on semiconductor chip 12. Moreover, hydrogen detection devices 10 and 10a each have a structure in which only the first resistive element (hydrogen sensor 100) is exposed to hydrogen. Accordingly, in each of hydrogen detection devices 10 and 10a according to Embodiment 1 and the variation thereof each including the bridge circuit, in a hydrogen-free environment, the four resistive elements show resistance values that are very close to each other, and in a hydrogen-containing environment, the resistance balance in the bridge circuit is disrupted and a potential difference is generated between two connection points. Thus, the hydrogen detection device that can operate stably and does not necessarily require a heater is realized.

Moreover, since each of hydrogen detection devices 10 and 10a according to Embodiment 1 and the variation thereof includes the four resistive elements that have basically the same layered structure, each of hydrogen detection devices 10 and 10a has extremely small temperature dependence of an output voltage and can be realized in a smaller size compared to hydrogen detection device 90 according to the reference example that includes a fixed resistor.

Moreover, in hydrogen detection device 10a according to the variation of Embodiment 1, in the second resistive element, the third resistive element, and the fourth resistive element, insulating films 107b and the like include openings 110a to 110c each of which is not covered by insulating film 107b and the like and through which an other surface of second electrode 106 opposite to the principal surface of second electrode 106 is exposed, openings 110a to 110c including inner side surfaces and bottom surfaces covered by hydrogen impermeable films 111a to 111c. Accordingly, an opening can be formed in the same manufacturing process for any of the four resistive elements, the structures of the four resistive elements including the openings can be made uniform, and the bridge circuit with extremely high precision can be realized without significantly increasing the number of manufacturing steps.

Moreover, the first resistive element, the second resistive element, the third resistive element, and the fourth resistive element form a rectangular shape in the plan view of semiconductor substrate 102 and are arranged at positions corresponding to four sides of the rectangular shape. Accordingly, high-density integration of the four resistive elements on semiconductor substrate 102 enables miniaturization of the hydrogen detection device and enhancement of performance of the bridge circuit by equalizing the temperature environment.

Moreover, layered structure 106a and opening 110 of insulating film 107b and the like are arranged in a four-fold rotational symmetry pattern in the plan view of semiconductor substrate 102, and opening 110 of the first resistive element is positioned to result in rotational symmetry in the plan view of semiconductor substrate 102. Thus, since layered structure 106a and opening 110 of insulating film 107b and the like are arranged in a four-fold rotational symmetry pattern, the size of each of hydrogen detection devices 10d and 10e can be made small.

Moreover, a manufacturing method for manufacturing each of hydrogen detection devices 10 and 10a according to Embodiment 1 and the variation thereof is a manufacturing method for manufacturing a hydrogen detection device that includes a bridge circuit including a first resistive element (hydrogen sensor 100), a second resistive element (reference element 100a), a third resistive element (reference element 100b), and a fourth resistive element (reference element 100c). The manufacturing method includes: layered body forming step S10 that is forming, on semiconductor substrate 102, layered bodies for the first resistive element, the second resistive element, the third resistive element, and the fourth resistive element; and opening forming step S11 that is forming an opening in at least one of the layered bodies formed, wherein in layered body forming step S10, a layered body including: first electrode 103 including a principal surface and second electrode 106 including a principal surface, the principal surface of first electrode 103 and the principal surface of second electrode 106 facing each other; metal oxide layer 104 disposed in contact with the principal surface of first electrode 103 and the principal surface of second electrode 106; and insulating film 107b and the like that cover first electrode 103, second electrode 106, and metal oxide layer 104 is formed as each of the layered bodies for the first resistive element, the second resistive element, the third resistive element, and the fourth resistive element, and in opening forming step S11, opening 110 that is not covered by insulating film 107b and the like and through which an other surface of second electrode 106 opposite to the principal surface of second electrode 106 is exposed is formed in at least insulating film 107b and the like of the layered body for the first resistive element (hydrogen sensor 100).

Accordingly, the hydrogen detection device that can operate stably and does not necessarily require a heater can be manufactured. Moreover, since the layered bodies for the four resistive elements can be manufactured in a single process, the hydrogen detection device that can operate more stably and is smaller in size compared to hydrogen detection device 90 according to the reference example that includes a fixed resistor can be manufactured by a simplified manufacturing process.

### Embodiment 2

First, a hydrogen detection device according to Embodiment 2 will be described. The hydrogen detection device according to Embodiment 2 is different from Embodiment 1 in that, among four resistive elements included in a bridge circuit, two resistive elements arranged at opposing positions are hydrogen sensors, whereas in Embodiment 1, only one resistive element among the four resistive elements included in the bridge circuit is a hydrogen sensor. Hereinafter, description shall focus on the points of difference with Embodiment 1.

FIG. 10 is an equivalent circuit diagram of hydrogen detection device 10b according to Embodiment 2. In the present diagram, voltmeter 20 and DC voltage source 21 are also illustrated as external devices.

Hydrogen detection device 10b includes a bridge circuit including hydrogen sensor 100 that is an example of a first resistive element, reference element 100a that is an example of a second resistive element, reference element 100b that is an example of a third resistive element, and hydrogen sensor 200 that is an example of a fourth resistive element. Hydrogen detection device 10b has a configuration in which reference element 100c that is an example of the fourth resistive element of hydrogen detection device 10 according to Embodiment 1 is replaced with hydrogen sensor 200.

Hydrogen sensor 200 has exactly the same structure as hydrogen sensor 100. Namely, hydrogen sensor 200 includes the structure illustrated in FIG. 2A and FIG. 2B. Accordingly, similar to hydrogen sensor 100, hydrogen sensor 200 has a structure in which a layered body that is sensitive to hydrogen and disposed inside of hydrogen sensor 200 is exposed to the outside, and is therefore sensitive to hydrogen. Accordingly, in a hydrogen-free environment, the four resistive elements have the same resistance value. In a hydrogen-containing environment, only the resistance values of hydrogen sensors 100 and 200 decrease according to the hydrogen concentration.

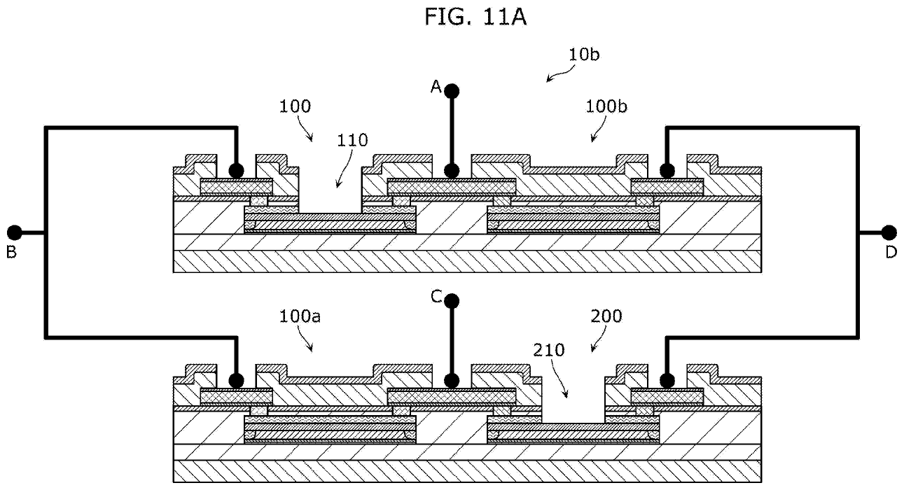

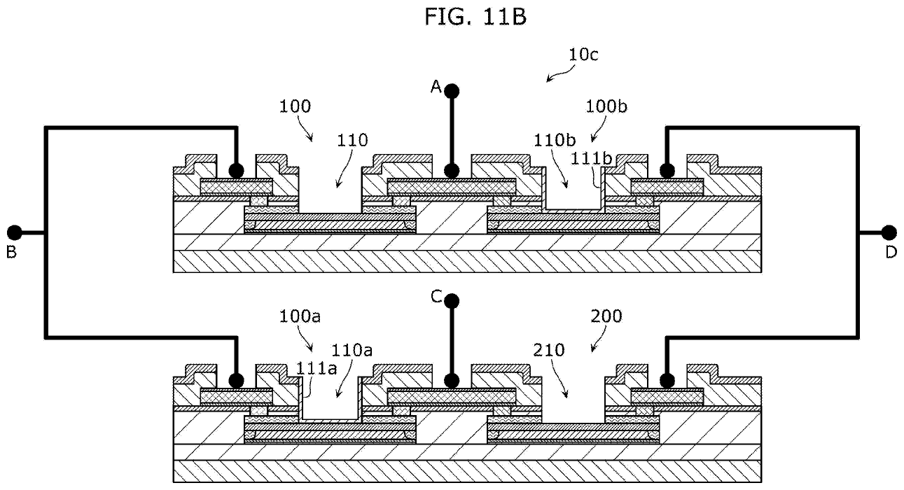

FIG. 11A is a schematic cross-sectional view of an example of an overall configuration of hydrogen detection device 10b according to Embodiment 2. As illustrated in the present diagram, the feature of hydrogen detection device 10b is that the four resistive elements (hydrogen sensor 100, reference element 100a, reference element 100b, and hydrogen sensor 200) included in the bridge circuit are provided on semiconductor chip 12, and in particular, that the spacing between the four resistive elements that have basically the same layered structure except for openings 110 and 210 in hydrogen sensors 100 and 200 is less than or equal to 2000 μm in plan view.

First terminal TE1 (i.e., one end) of hydrogen sensor 100 and first terminal TE1 (i.e., one end) of reference element 100a are connected as terminal B, first terminal TE1 (i.e., one end) of reference element 100b and first terminal TE1 (i.e., one end) of hydrogen sensor 200 are connected as terminal D, second terminal TE2 (i.e., the other end) of hydrogen sensor 100 and second terminal TE2 (i.e., the other end) of reference element 100b are connected as terminal A, and second terminal TE2 (i.e., the other end) of reference element 100a and second terminal TE2 (i.e., the other end) of hydrogen sensor 200 are connected as terminal C.

FIG. 11B is a schematic view illustrating an example of an overall configuration of hydrogen detection device 10c according to a variation of Embodiment 2. The difference from hydrogen detection device 10b according to Embodiment 2 illustrated in FIG. 11A is that, in hydrogen detection device 10c according to the present variation, openings 110a and 110b similar to openings 110 and 210 of hydrogen sensors 100 and 200 are formed in reference elements 100a and 100b in the same manufacturing process and then the inner side surfaces and the bottom surfaces of openings 110a and 110b are covered by hydrogen impermeable films 111a and 111b.

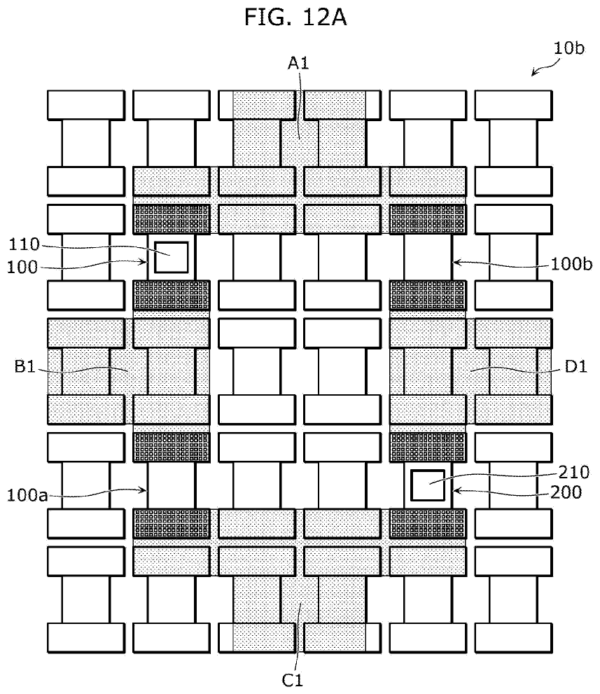

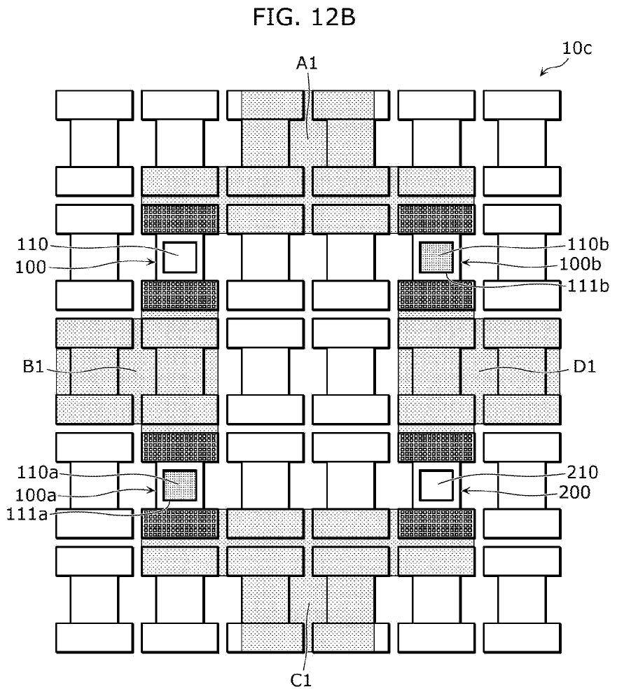

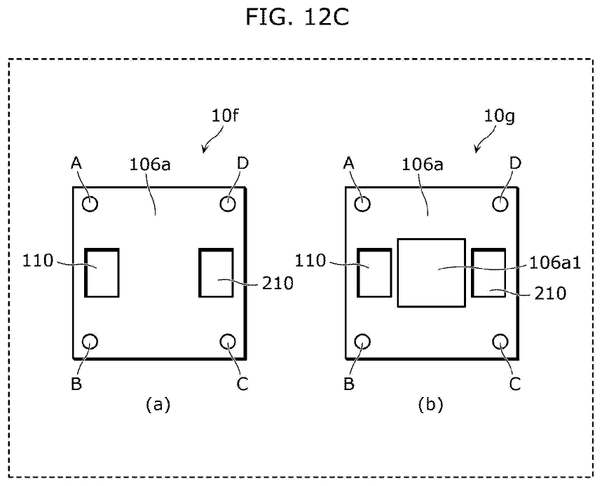

FIG. 12A is a plan view illustrating an example of the layout of wiring patterns of the four resistive elements in hydrogen detection device 10b according to Embodiment 2 illustrated in FIG. 11A. The present plan view illustrates: wiring pattern A1 that connects second terminal TE2 of hydrogen sensor 100 including opening 110 and second terminal TE2 of reference element 100b without an opening, and connects to terminal A (not illustrated) of the bridge circuit; wiring pattern B1 that connects first terminal TE1 of hydrogen sensor 100 including opening 110 and first terminal TE1 of reference element 100a without an opening, and connects to terminal B (not illustrated) of the bridge circuit; wiring pattern C1 that connects second terminal TE2 of reference element 100a without an opening and second terminal TE2 of hydrogen sensor 200 including opening 210, and connects to terminal C (not illustrated) of the bridge circuit; and wiring pattern D1 that connects first terminal TE1 of hydrogen sensor 200 including opening 210 and first terminal TE1 of reference element 100b without an opening, and connects to terminal D (not illustrated) of the bridge circuit.

FIG. 12B is a plan view illustrating an example of the layout of the four resistive elements in hydrogen detection device 10c according to the variation of Embodiment 2 illustrated in FIG. 11B. The present plan view illustrates: wiring pattern A1 that connects second terminal TE2 of hydrogen sensor 100 including opening 110 and second terminal TE2 of reference element 100b including opening 110b covered by hydrogen impermeable film 111b, and connects to terminal A (not illustrated) of the bridge circuit; wiring pattern B1 that connects first terminal TE1 of hydrogen sensor 100 including opening 110 and first terminal TE1 of reference element 100a including opening 110a covered by hydrogen impermeable film 111a, and connects to terminal B (not illustrated) of the bridge circuit; wiring pattern C1 that connects second terminal TE2 of reference element 100a including opening 110a covered by hydrogen impermeable film 111a and second terminal TE2 of hydrogen sensor 200 including opening 210, and connects to terminal C (not illustrated) of the bridge circuit; and wiring pattern D1 that connects first terminal TE1 of hydrogen sensor 200 including opening 210 and first terminal TE1 of reference element 100b including opening 110b covered by hydrogen impermeable film 111b, and connects to terminal D (not illustrated) of the bridge circuit.

In any of the layout examples of FIG. 12A and FIG. 12B, the four resistive elements form a rectangular shape in the plan view of semiconductor substrate 102 and are arranged at positions corresponding to the four sides of the rectangular shape, and by the high-density integration, it is possible to reduce the size of the hydrogen detection device and to enhance the performance of the bridge circuit by unifying the temperature environment.

FIG. 12C is a plan view illustrating the rotational symmetry of openings 110 and 210 of insulating film 107b and the like and the rotational symmetry of layered structure 106a and the like in each of hydrogen detection devices 10f and 10g according to the variation of Embodiment 2. More specifically, (a) of FIG. 12C illustrates the rotational symmetry of openings 110 and 210 of insulating film 107b and the like and the rotational symmetry of layered structure 106a and the like when layered structure 106a and the like do not include an opening, and in contrast, (b) of FIG. 12C illustrates the rotational symmetry of openings 110 and 210 of insulating film 107b and the like and the rotational symmetry of layered structure 106a and the like when layered structure 106a and the like are films that include, at their center, opening 106a1 that is an electrically insulated area. It should be noted that FIG. 12C corresponds to a variation of the layout examples illustrated in FIG. 12A and FIG. 12B. Moreover, layered structure 106a and the like are layered structure films (103, 104, and 106) included in a sensor element, and insulating film 107b and the like are insulating films (insulating films 107a to 107c, 109a, and 109b) that cover hydrogen sensor 100.

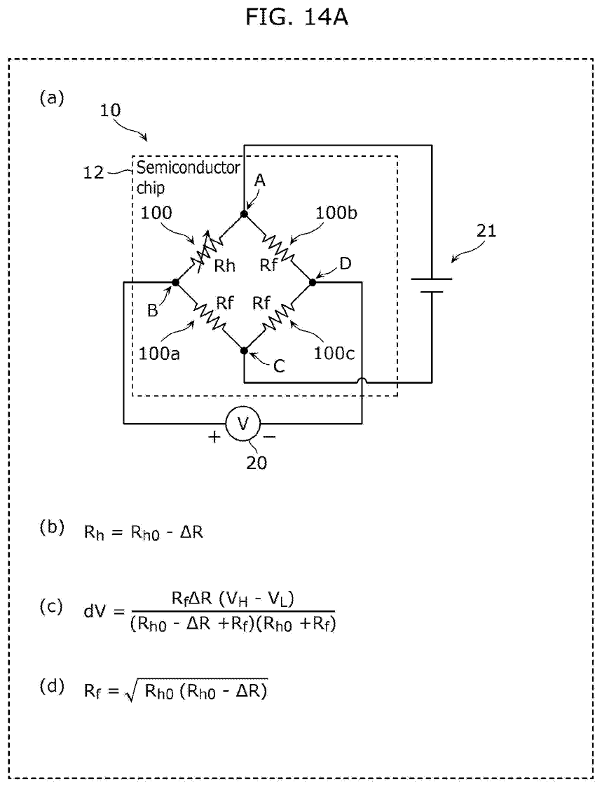

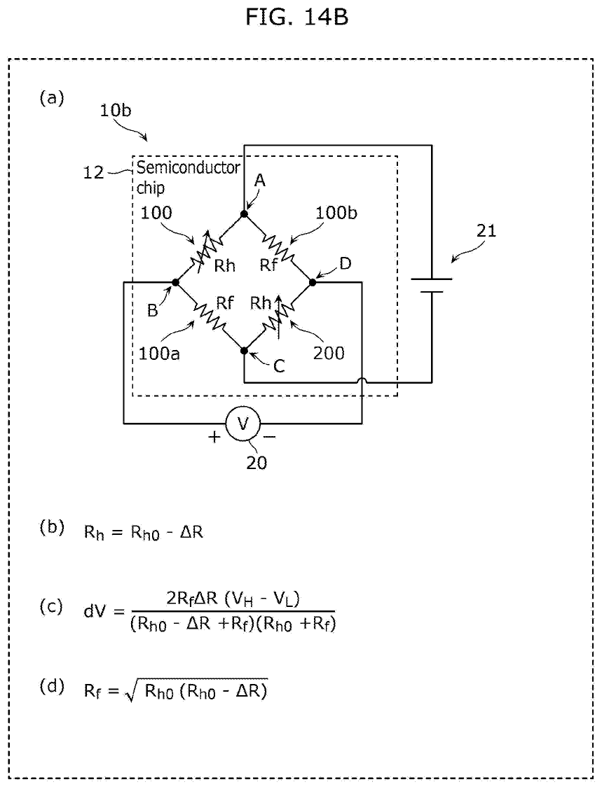

In any of (a) and (b) of FIG. 12C, layered structure 106a and openings 110 and 210 of insulating film 107b and the like are arranged in a two-fold rotational symmetry pattern (i.e., a rotationally symmetric pattern with 2-fold symmetry) in the plan view of semiconductor substrate 102. Thus, since layered structure 106a and openings 110 and 210 of insulating film 107b and the like are arranged in a two-fold rotational symmetry pattern, the size of each of hydrogen detection devices 10f and 10g can be made small and, as described later in FIG. 14B, hydrogen can be detected with twice the sensitivity of each of hydrogen detection devices 10d and 10e according to Embodiment 1 and the variation thereof.

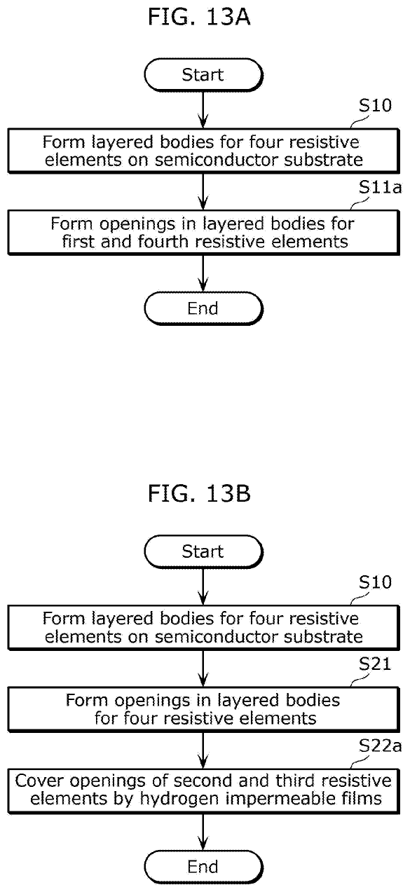

FIG. 13A is a flowchart illustrating a manufacturing method for manufacturing hydrogen detection device 10b according to Embodiment 2. The difference from the manufacturing method for manufacturing hydrogen detection device 10 according to Embodiment 1 illustrated in FIG. 6A is that, in the second step (S11a), instead of forming an opening in the layered body for the first resistive element (S11), an opening is formed in each of the layered bodies for the first and fourth resistive elements (S11a).

In other words, in the second step (S11a), photolithography (pattern transferring and etching) is performed on each of the layered bodies for hydrogen sensors 100 and 200 produced in the first step (S10) to remove, in a rectangular shape, part of metal layer 106s, insulating film 107b, insulating film 109a, insulating film 107c, and insulating film 109b so that at least part of the upper surface of second electrode 106 is exposed, and thus opening 110 of hydrogen sensor 100 and opening 210 of hydrogen sensor 200 are formed (opening forming step S11a). By this step, the manufacture of hydrogen detection device 10b is completed.

FIG. 13B is a flowchart illustrating a manufacturing method for manufacturing hydrogen detection device 10c according to the variation of Embodiment 2. The difference from the manufacturing method for manufacturing hydrogen detection device 10a according to the variation of Embodiment 1 illustrated in FIG. 6B is that, in the third step (S22a), instead of covering the openings for the second to fourth resistive elements by hydrogen impermeable films (S22), the openings for the second and third resistive elements are covered by hydrogen impermeable films (S22a).

Namely, in the third step (S22a), the inner side surfaces and the bottom surfaces of the openings for the second and third resistive elements (i.e., openings 110a and 110b formed for reference elements 100a and 100b) are covered by hydrogen impermeable films 111a and 111b each containing, for example, P-SiON (hydrogen impermeable film forming step S22a). By this step, the manufacture of reference elements 100a and 100b provided with openings 110a and 110b including the inner side surfaces and the bottom surfaces covered by hydrogen impermeable films 111a and 111b is completed. It should be noted that a process that is the same as the process of forming insulating film 109b (i.e., film formation using the same material as that of insulating film 109b) may be performed for forming hydrogen impermeable films 111a and 111b. By this step, the manufacture of hydrogen detection device 10c according to the variation of Embodiment 2 is completed.

FIG. 14A is a diagram for describing differential voltage change amount dV of hydrogen detection device 10 according to Embodiment 1. (a) of FIG. 14A illustrates a bridge circuit configuration of hydrogen detection device 10 according to Embodiment 1, (b) of FIG. 14A shows resistance value Rh of hydrogen sensor 100 when exposed to hydrogen, (c) of FIG. 14A shows differential voltage change amount dV (strictly, the maximum value of differential voltage change amount dV) of hydrogen detection device 10 when exposed to hydrogen, and (d) of FIG. 14A shows a conditional expression for maximizing differential voltage change amount dV shown in (c) of FIG. 14A.

In FIG. 14A, Rh denotes a resistance value of hydrogen sensor 100, Rf denotes a resistance value of each of reference elements 100a, 100b, and 100c, Rh0 denotes a resistance value of hydrogen sensor 100 when there is no hydrogen, ΔR denotes decrease (change) in resistance of hydrogen sensor 100 when exposed to hydrogen, and differential voltage change amount dV in (c) of FIG. 14A denotes a change amount of differential voltage between terminal B and terminal D when exposed to hydrogen.

As shown in (b) of FIG. 14A, by exposure to hydrogen, resistance value Rh of hydrogen sensor 100 is decreased from resistance value Rh0 by change amount ΔR. As a result, differential voltage change amount dV when exposed to hydrogen is given by the equation shown in (c) of FIG. 14A.

FIG. 14B is a diagram for describing differential voltage change amount dV of hydrogen detection device 10b according to Embodiment 2. It should be noted that the content shown in the present diagram also applies to hydrogen detection device 10c according to the variation of Embodiment 2.

(a) of FIG. 14B illustrates a bridge circuit configuration of hydrogen detection device 10b according to Embodiment 2, (b) of FIG. 14B shows resistance value Rh of each of hydrogen sensors 100 and 200 when exposed to hydrogen, (c) of FIG. 14B shows differential voltage change amount dV (strictly, the maximum value of differential voltage change amount dV) of hydrogen detection device 10b when exposed to hydrogen, and (d) of FIG. 14B shows a conditional expression for maximizing differential voltage change amount dV shown in (c) of FIG. 14B.

In FIG. 14B, Rh denotes a resistance value of each of hydrogen sensors 100 and 200, Rf denotes a resistance value of each of reference elements 100a and 100b, Rh0 denotes a resistance value of each of hydrogen sensors 100 and 200 when there is no hydrogen, ΔR denotes decrease (change) in resistance of each of hydrogen sensors 100 and 200 when exposed to hydrogen, and differential voltage change amount dV in (c) of FIG. 14B denotes a change amount of differential voltage between terminal B and terminal D when exposed to hydrogen.

As shown in (b) of FIG. 14B, by exposure to hydrogen, resistance value Rh of each of hydrogen sensors 100 and 200 is decreased by change amount ΔR from resistance value Rh0. As a result, differential voltage change amount dV when exposed to hydrogen is given by the equation shown in (c) of FIG. 14B.

Here, as can be seen from a comparison between (c) of FIG. 14A and (c) of FIG. 14B, differential voltage change amount dV of hydrogen detection device 10b according to Embodiment 2 ((b) of FIG. 14B) is twice as large as differential voltage change amount dV of hydrogen detection device 90 according to the reference example ((b) of FIG. 14A). Here, differential voltage change amount dV of hydrogen detection device 90 according to the reference example is the same as differential voltage change amount dV of each of hydrogen detection devices 10 and 10a according to Embodiment 1 and the variation thereof, because their bridge circuits are identical. Accordingly, hydrogen detection device 10b according to Embodiment 2 can detect hydrogen with twice the sensitivity of each of hydrogen detection device 90 according to the reference example and hydrogen detection devices 10 and 10a according to Embodiment 1 and the variation thereof.

As described above, each of hydrogen detection devices 10b and 10c according to Embodiment 2 and the variation thereof includes: a bridge circuit including a first resistive element (hydrogen sensor 100), a second resistive element (reference element 100a), a third resistive element (reference element 100b), and a fourth resistive element (reference element 100c), wherein one end of the first resistive element and one end of the second resistive element are connected to each other, one end of the third resistive element and one end of the fourth resistive element are connected to each other, an other end of the first resistive element and an other end of the third resistive element are connected to each other, an other end of the second resistive element and an other end of the fourth resistive element are connected to each other, each of the first resistive element, the second resistive element, the third resistive element, and the fourth resistive element is provided on semiconductor substrate 102 and includes: first electrode 103 including a principal surface and second electrode 106 including a principal surface, the principal surface of first electrode 103 and the principal surface of second electrode 106 facing each other; metal oxide layer 104 disposed in contact with the principal surface of first electrode 103 and the principal surface of second electrode 106; and insulating film 107b and the like covering first electrode 103, second electrode 106, and metal oxide layer 104, and in at least the first resistive element among the first resistive element, the second resistive element, the third resistive element, and the fourth resistive element, insulating film 107b and the like include opening 110 that is not covered by insulating film 107b and the like and through which an other surface of second electrode 106 opposite to the principal surface of second electrode 106 is exposed. Here, further in the fourth resistive element, insulating film 107b and the like include opening 210 that is not covered by insulating film 107b and the like and through which an other surface of second electrode 106 opposite to the principal surface of second electrode 106 is exposed.

Accordingly, the four resistive elements included in the bridge circuit are resistive elements that have basically the same layered structure, and are provided on semiconductor chip 12. Moreover, hydrogen detection devices 10b and 10c each have a structure in which only the first resistive element (hydrogen sensor 100) and the fourth resistive element (hydrogen sensor 200) are exposed to hydrogen. Accordingly, similar to hydrogen detection devices 10 and 10a according to Embodiment 1 and the variation thereof, hydrogen detection devices 10b and 10c according to Embodiment 2 and the variation thereof each including a bridge circuit do not necessarily require a heater and can operate stably, and have extremely small temperature dependence of an output voltage and can be realized in a smaller size compared to hydrogen detection device 90 according to the reference example that includes a fixed resistor.

Moreover, hydrogen detection devices 10b and 10c according to Embodiment 2 and the variation thereof can detect hydrogen with twice the sensitivity of each of hydrogen detection devices 10 and 10a according to Embodiment 1 and the variation thereof.

Moreover, in the second resistive element and the third resistive element, insulating films 107b and the like include openings 110a and 110b each of which is not covered by insulating film 107b and the like and through which an other surface of second electrode 106 opposite to the principal surface of second electrode 106 is exposed, openings 110a and 110b including inner side surfaces and bottom surfaces that are covered by hydrogen impermeable films 111a and 111b. Accordingly, an opening can be formed in the same manufacturing process for any of the four resistive elements, the structures of the four resistive elements including the openings can be made uniform, and a bridge circuit with extremely high precision can be realized without significantly increasing the number of manufacturing steps.

Moreover, the first resistive element, the second resistive element, the third resistive element, and the fourth resistive element form a rectangular shape in the plan view of semiconductor substrate 102 and are arranged at positions corresponding to four sides of the rectangular shape. Accordingly, high-density integration of the four resistive elements on semiconductor substrate 102 enables miniaturization of the hydrogen detection device and enhancement of performance of the bridge circuit by equalizing the temperature environment.

Moreover, layered structure 106a and the like and openings 110 and 210 of insulating film 107b and the like are arranged in a two-fold rotational symmetry pattern in the plan view of semiconductor substrate 102. Accordingly, since layered structure 106a and the like and insulating film 107b and the like including openings 110 and 210 are arranged in a two-fold rotational symmetry pattern, the size of each of hydrogen detection devices 10b and 10c can be made small and hydrogen can be detected with twice the sensitivity of each of hydrogen detection devices 10d and 10e according to Embodiment 1 and the variation thereof. In this case, an electrically insulated area may be provided at the center of the first to fourth resistive elements forming the rectangular shape.

Moreover, a manufacturing method for manufacturing each of hydrogen detection devices 10b and 10c according to Embodiment 2 and the variation thereof is a manufacturing method for manufacturing a hydrogen detection device that includes a bridge circuit including a first resistive element (hydrogen sensor 100), a second resistive element (reference element 100a), a third resistive element (reference element 100b), and a fourth resistive element (hydrogen sensor 200). The manufacturing method includes: layered body forming step S10 that is forming, on semiconductor substrate 102, layered bodies for the first resistive element, the second resistive element, the third resistive element, and the fourth resistive element; and opening forming step S11a that is forming an opening in at least one of the layered bodies formed, wherein in layered body forming step S10, a layered body including: first electrode 103 including a principal surface and second electrode 106 including a principal surface, the principal surface of first electrode 103 and the principal surface of second electrode 106 facing each other; metal oxide layer 104 disposed in contact with the principal surface of first electrode 103 and the principal surface of second electrode 106; and insulating film 107b and the like that cover first electrode 103, second electrode 106, and metal oxide layer 104 is formed as each of the layered bodies for the first resistive element, the second resistive element, the third resistive element, and the fourth resistive element, and in opening forming step S11a, opening 110 that is not covered by insulating film 107b and the like and through which an other surface of second electrode 106 opposite to the principal surface of second electrode 106 is exposed is formed in at least insulating film 107b and the like of the layered body for the first resistive element (hydrogen sensor 100). Here, in opening forming step S11a, opening 210 that is not covered by insulating film 107b and the like and through which an other surface of second electrode 106 opposite to the principal surface of second electrode 106 is exposed is further formed in insulating film 107b and the like of the layered body for the fourth resistive element (hydrogen sensor 200).

Accordingly, the hydrogen detection device that can operate stably and does not necessarily require a heater can be manufactured. Moreover, since the layered bodies for the four resistive elements can be manufactured in a single process, the hydrogen detection device that can operate more stably and is smaller in size compared to hydrogen detection device 90 according to the reference example that includes a fixed resistor can be manufactured by a simplified manufacturing process. Furthermore, the hydrogen detection device that can detect hydrogen with twice the sensitivity of each of hydrogen detection devices 10 and 10a according to Embodiment 1 and the variation thereof is manufactured.

Hereinabove, although the hydrogen detection device according to the present disclosure and the method for manufacturing the same have been described based on the embodiments and variations, the present disclosure is not limited to these embodiments and variations. Various modifications of the embodiments and variations as well as other embodiments resulting from combinations of some of the constituent elements from the embodiments and variations that may be conceived by those skilled in the art are included within the scope of the present disclosure as long as they do not depart from the essence of the present disclosure.

For example, in hydrogen detection device 10a according to the variation of Embodiment 1, openings 110a to 110c and hydrogen impermeable films 111a to 111c are provided for three reference elements 100a to 100c; however, it is not necessary to provide them for all three reference elements 100a to 100c, and an opening and a hydrogen impermeable film may be formed for at least one reference element among three reference elements 100a to 100c while an opening and a hydrogen impermeable film need not be formed for the rest of the three reference elements.

Likewise, in hydrogen detection device 10c according to the variation of Embodiment 2, openings 110a and 110b and hydrogen impermeable films 111a and 111b are provided for two reference elements 100a and 100b; however, it is not necessary to provide them for both two reference elements 100a and 100b, and an opening and a hydrogen impermeable film may be formed for one of two reference elements 100a or 100b while an opening and a hydrogen impermeable film need not be formed for the other of the two reference elements.

Moreover, although an example in which only a hydrogen detection device is provided on semiconductor chip 12 has been described in each of the above-described embodiments and variations, a circuit other than the hydrogen detection device, for example, a buffer amplifier that amplifies a differential voltage outputted from the bridge circuit, a constant voltage power supply circuit that generates a voltage to be applied to the bridge circuit, or the like may also be provided on semiconductor chip 12.

### INDUSTRIAL APPLICABILITY

A hydrogen detection device according to the present disclosure can be used as a hydrogen detection device that operates stably with high sensitivity using a bridge circuit, for example, as a hydrogen detection device that is provided to a fuel-cell vehicle.

1. A hydrogen detection device comprising:
a bridge circuit including a first resistive element, a second resistive element, a third resistive element, and a fourth resistive element, wherein
one end of the first resistive element and one end of the second resistive element are connected to each other,
one end of the third resistive element and one end of the fourth resistive element are connected to each other,
an other end of the first resistive element and an other end of the third resistive element are connected to each other,
an other end of the second resistive element and an other end of the fourth resistive element are connected to each other,
each of the first resistive element, the second resistive element, the third resistive element, and the fourth resistive element is provided on a semiconductor substrate and includes:
a first electrode including a principal surface and a second electrode including a principal surface, the principal surface of the first electrode and the principal surface of the second electrode facing each other;
a metal oxide layer disposed in contact with the principal surface of the first electrode and the principal surface of the second electrode; and
an insulating film covering the first electrode, the second electrode, and the metal oxide layer, and
in at least the first resistive element among the first resistive element, the second resistive element, the third resistive element, and the fourth resistive element, the insulating film includes an opening that is not covered by the insulating film and through which an other surface of the second electrode opposite to the principal surface of the second electrode is exposed.

2. The hydrogen detection device according to claim 1, wherein
in each of the second resistive element, the third resistive element, and the fourth resistive element, the insulating film includes an opening that is not covered by the insulating film and through which an other surface of the second electrode opposite to the principal surface of the second electrode is exposed, the opening including an inner side surface and a bottom surface that are covered by a hydrogen impermeable film.

3. The hydrogen detection device according to claim 1, wherein
further in the fourth resistive element, the insulating film includes an opening that is not covered by the insulating film and through which an other surface of the second electrode opposite to the principal surface of the second electrode is exposed.

4. The hydrogen detection device according to claim 3, wherein
in each of the second resistive element and the third resistive element, the insulating film includes an opening that is not covered by the insulating film and through which an other surface of the second electrode opposite to the principal surface of the second electrode is exposed, the opening including an inner side surface and a bottom surface that are covered by a hydrogen impermeable film.

5. The hydrogen detection device according to claim 1, wherein
the first resistive element, the second resistive element, the third resistive element, and the fourth resistive element form a rectangular shape in plan view of the semiconductor substrate and are arranged at positions corresponding to four sides of the rectangular shape.

6. The hydrogen detection device according to claim 5, wherein
the first to fourth resistive elements forming the rectangular shape are arranged in a four-fold rotational symmetry pattern in the plan view of the semiconductor substrate, and
the opening in the first resistive element is positioned to result in four-fold rotational symmetry in the plan view of the semiconductor substrate.

7. The hydrogen detection device according to claim 5, wherein
the first to fourth resistive elements forming the rectangular shape and the insulating film including the opening in each of the first resistive element and the fourth resistive element are arranged in a two-fold rotational symmetry pattern in the plan view of the semiconductor substrate.

8. The hydrogen detection device according to claim 5, wherein
an electrically insulated area is provided at a center of the first to fourth resistive elements forming the rectangular shape.

9. A manufacturing method for manufacturing a hydrogen detection device that includes a bridge circuit including a first resistive element, a second resistive element, a third resistive element, and a fourth resistive element, the manufacturing method comprising:
forming, on a semiconductor substrate, layered bodies for the first resistive element, the second resistive element, the third resistive element, and the fourth resistive element; and
forming an opening in at least one of the layered bodies formed, wherein
in the forming of layered bodies, a layered body including: a first electrode including a principal surface and a second electrode including a principal surface, the principal surface of the first electrode and the principal surface of the second electrode facing each other; a metal oxide layer disposed in contact with the principal surface of the first electrode and the principal surface of the second electrode; and an insulating film that covers the first electrode, the second electrode, and the metal oxide layer is formed as each of the layered bodies for the first resistive element, the second resistive element, the third resistive element, and the fourth resistive element, and
in the forming of an opening, an opening that is not covered by the insulating film and through which an other surface of the second electrode opposite to the principal surface of the second electrode is exposed is formed in at least the insulating film of the layered body for the first resistive element.

10. The manufacturing method according to claim 9, wherein
in the forming of an opening, an opening that is not covered by the insulating film and through which an other surface of the second electrode opposite to the principal surface of the second electrode is exposed is further formed in the insulating film of the layered body for the fourth resistive element.
